(19) United States
(12) Patent Application Publication
(10) Pub. No.: US 2023/0191881 A1
(43) Pub. Date: Jun. 22, 2023

(54) SCENT DIFFUSION CARTRIDGE CONVERSION SYSTEM AND METHOD FOR VEHICLE

(71) Applicants: Hyundai Motor Company, Seoul (KR); Kia Corporation, Seoul (KR); Hanon Systems, Daejon (KR)

(72) Inventors: Myung Jun Kim, Busan (KR); In Taek Park, Hwaseong (KR); Tae Kun Kim, Ansan (KR); Dong Won Yeon, Daejeon (KR); Ei Hyun Choi, Daejeon (KR); Hong Jae Kim, Seoul (KR); Jun Sang Rhee, Seoul (KR)

(21) Appl. No.: 18/074,888

(22) Filed: Dec. 5, 2022

(30) Foreign Application Priority Data
Dec. 21, 2021 (KR) 10-2021-0183789

(51) Int. Cl. B60H 3/00

(52) U.S. Cl. CPC B60H 3/0035; B60H 2003/0064

(57) ABSTRACT

The present disclosure comprises a plurality of scent diffusion cartridge main body units configured to generate different scents, a mounting recognition unit comprising a plurality of mounting regions in which the scent diffusion cartridge main body unit is mounted, configured to detect whether the scent diffusion cartridge main body unit is mounted in the mounting region, and a control unit configured to determine whether the scent diffusion cartridge main body unit is mounted in the mounting region from the mounting recognition unit, and control a scent of the scent diffusion cartridge main body unit mounted in another mounting region to be selectively diffused as the scent diffusion cartridge main body unit is dismounted from the mounting region.

### CROSS-REFERENCE TO RELATED APPLICATION(S)

This application claims, under 35 U.S.C. § 119(a), the benefit of Korean Patent Application No. 10-2021-0183789, filed on Dec. 21, 2021, the disclosure of which is incorporated herein by reference in its entirety.

### BACKGROUND

### Technical Field

Embodiments of the present disclosure relate to a scent diffusion cartridge conversion system and method for a vehicle, and more specifically, to a scent diffusion cartridge conversion system and method for a vehicle, configured to automatically convert a currently operating scent diffusion cartridge into the other scent diffusion cartridge when the currently operating scent diffusion cartridge of a plurality of scent diffusion cartridges is removed or damaged, thereby providing convenience to a driver while traveling.

### Background

In general, air conditioners for a vehicle are defined as a device for cooling or heating a vehicle interior by heating or cooling in a process of introducing air outside the vehicle into a vehicle interior or circulating the air in the vehicle interior.

These air conditioners for a vehicle are configured to blow air into an air-conditioning case via a blower unit, and are provided with an evaporator configured for performing a cooling operation and a heater core configured for performing a heating operation inside the case, and are configured so that the air cooled or heated by the evaporator or the heater core is selectively blown to each portion of the vehicle interior using a blowing mode conversion door and the like.

Recently, various harmful substances such as dust or soot gas generated from the road have been introduced into vehicle interiors when air conditioners are operated while vehicles are traveling, and in addition, bacterial foreign substances such as fungi living in the air conditioning ducts have been directly introduced into vehicle interiors as the air conditioners are operated in wet weather, so that scent generators for purifying the air inside the vehicles have been installed and used.

This scent generator is typically configured to discharge a specific single scent, but there is a need for the development for a device capable of allowing a desired scent of two scents to be discharged in order to respect individual preference. However, in this case, there is a problem in that, when an abnormal situation such as damage or separation of the selected and operating scent diffusion cartridge occurs, the corresponding function may not be provided to the driver while driving.

In addition, there is a problem in that, when the difference in the degree of scent diffusion of the two scents occurs over time, or when a situation in which the scent cartridge for scent diffusion is damaged or is not mounted occurs, it is difficult to be automatically replaced with or converted into another scent, and thus the corresponding function may not be provided to the driver while driving.

The above information disclosed in this Background section is only for enhancement of understanding of the background of the present disclosure and, accordingly, it may comprise information that does not form the existing technologies that are already known in this country to a person of ordinary skill in the art.

### SUMMARY

An object of the present disclosure is to provide a scent diffusion cartridge conversion system and method for a vehicle, in which a plurality of scent diffusion cartridges may be provided, but a currently operating scent diffusion cartridge may be automatically converted into the other scent diffusion cartridge mounted when the currently operating scent diffusion cartridge is removed or damaged, for example, when the scent of the current scent diffusion cartridge is intended to be changed, an automatic conversion into a scent of the other scent diffusion cartridge and a change in the type of scent may be performed only when the currently operating scent diffusion cartridge is removed even without operation of a separate audio video navigation (AVN) for changing, thereby providing convenience according to the conversion of the scent diffusion cartridge to the driver while traveling.

A scent diffusion cartridge conversion system for a vehicle according to the present disclosure may comprise: a plurality of scent diffusion cartridge main body units configured to generate different scents, a mounting recognition unit comprising a plurality of mounting regions in which the scent diffusion cartridge main body unit is mounted, configured to detect whether the scent diffusion cartridge main body unit is mounted in the mounting region, and a control unit configured to determine whether the scent diffusion cartridge main body unit is mounted in the mounting region from the mounting recognition unit, and control a scent of the scent diffusion cartridge main body unit mounted in another mounting region to be selectively diffused as the scent diffusion cartridge main body unit is dismounted from the mounting region.

The control unit may be configured to control scent information of the scent diffusion cartridge main body unit mounted in the mounting region to be displayed respectively according to a type of scent, and control the scent information to be selectively convertible by a user's operation.

The control unit may be configured to control a selection of a corresponding scent diffusion cartridge main body unit dismounted to be inactivated as the scent diffusion cartridge main body unit is converted from a mounted state into a dismounted state in the mounting region.

The control unit may be configured to control a selection of all of the scent diffusion cartridge main body units to be inactivated as all of the scent diffusion cartridge main body units are converted from a mounted state into a dismounted state in the mounting region.

For scent diffusion cartridge main body units corresponding to a mounted state to be activated, the control unit may be configured to control a number of scent diffusion cartridge main body unit mounted in the mounting region to be selectively displayed.

A method for scent diffusion cartridge conversion for a vehicle according to the present disclosure may comprise: determining, for a plurality of scent diffusion cartridge main body units mounted in a plurality of mounting regions provided in a mounting recognition unit, whether each of the plurality of scent diffusion cartridge main body units is mounted, controlling a type of scent of any one scent diffusion cartridge main body unit, of the plurality of scent diffusion cartridge main body units, as all of the scent diffusion cartridge main body units are mounted in the mounting region, determining whether each of the plurality of scent diffusion cartridge main body units is dismounted, and controlling a scent of each scent diffusion cartridge main body unit mounted in another mounting region to be diffused as it is determined that a mounted scent diffusion cartridge main body unit is dismounted.

According to various embodiments, the controlling the scent further comprises controlling a selection of a scent diffusion cartridge main body unit to be inactivated as it is determined that all of the plurality of scent diffusion cartridge main body units are dismounted from the mounting region.

According to various embodiments, the determining whether each of the plurality of scent diffusion cartridge main body units is mounted further comprises determining a number of mounted scent diffusion cartridge main body units, and controlling a number of scent diffusion cartridge main body units mounted in the mounting region to be displayed as it is determined that all of the scent diffusion cartridge main body units are not mounted in the mounting region.

According to various embodiments, The operation of determining the number of mounted scent diffusion cartridge main body units controls the selection of the scent diffusion cartridge main body unit in a normal state to be selectively activated as it is determined that the number of scent diffusion cartridge main body units mounted in the mounting region is not 0.

According to various embodiments, the operation of determining the number of mounted scent diffusion cartridge main body units controls the selection of the scent diffusion cartridge main body unit in a normal state to be inactivated as it is determined that the number of scent diffusion cartridge main body units mounted in the mounting region is 0.

According to the present disclosure, a plurality of scent diffusion cartridges may be provided, but the currently operating scent diffusion cartridge may be automatically converted into the other scent diffusion cartridge mounted when the currently operating scent diffusion cartridge is removed or damaged, for example, when the scent of the current scent diffusion cartridge is intended to be changed, the automatic conversion into the scent of the other scent diffusion cartridge and the change in the type of scent may be performed only when the currently operating scent diffusion cartridge is removed even without operation of the separate audio video navigation (AVN) for changing, thereby providing convenience according to the conversion of the scent diffusion cartridge to the driver while traveling.

In addition, according to the present disclosure, when it is determined that all of the plurality of scent diffusion cartridges are removed or damaged, the scent diffusion intensity can be controlled to be OFF, and at the same time, the selection of the scent diffusion cartridge may also be controlled to be inactivated on the display unit of the vehicle, thereby preventing the occurrence of the unnecessary selection operation in advance.

It is understood that the term “automotive” or “vehicular” or other similar term as used herein is inclusive of motor automotives in general such as passenger automobiles including sports utility automotives (operation SUV), buses, trucks, various commercial automotives, watercraft including a variety of boats and ships, aircraft, and the like, and includes hybrid automotives, electric automotives, plug-in hybrid electric automotives, hydrogen-powered automotives and other alternative fuel automotives (e.g., fuels derived from resources other than petroleum). As referred to herein, a hybrid automotive is an automotive that has two or more sources of power, for example both gasoline-powered and electric-powered automotives.

The above and other features of the disclosure are discussed infra.

### BRIEF DESCRIPTION OF THE DRAWINGS

The above and other features of the present disclosure will now be described in detail with reference to certain exemplary examples thereof illustrated in the accompanying drawings which are given herein below by way of illustration only, and thus are not limitative of the present disclosure, and wherein:

FIG. 1 is a diagram showing a configuration of a scent diffusion cartridge conversion system for a vehicle according to an exemplary embodiment of the present disclosure.

FIG. 2 is a diagram showing a scent diffusion cartridge main body unit of the scent diffusion cartridge conversion system for a vehicle according to an exemplary embodiment of the present disclosure.

FIG. 3 is a diagram showing a display unit of the scent diffusion cartridge conversion system for a vehicle according to an exemplary embodiment of the present disclosure.

FIG. 4 is a diagram schematically showing the scent diffusion cartridge conversion system for a vehicle according to an exemplary embodiment of the present disclosure.

FIG. 5 is a diagram sequentially showing a scent diffusion cartridge conversion method for a vehicle according to another embodiment of the present disclosure.

FIG. 6 is a diagram showing a selection screen of the scent diffusion cartridge main body unit in the scent diffusion cartridge conversion method for a vehicle according to another embodiment of the present disclosure.

FIG. 7 is a diagram showing another selection screen of the scent diffusion cartridge main body unit in the scent diffusion cartridge conversion method for a vehicle according to another embodiment of the present disclosure.

FIG. 8 is a diagram showing a screen in which the selection of the scent diffusion cartridge main body unit in the scent diffusion cartridge conversion method for a vehicle according to another embodiment of the present disclosure is inactivated.

It should be understood that the appended drawings are not necessarily to scale, presenting a somewhat simplified representation of various preferred features illustrative of the basic principles of the disclosure. The specific design features of the present disclosure as disclosed herein, including, for example, specific dimensions, orientations, locations, and shapes will be determined in section by the particular intended application and use environment.

In the figures, reference numbers refer to the same or equivalent sections of the present disclosure throughout the several figures of the drawing.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Hereinafter, preferred embodiments according to the present disclosure will be described in detail with reference to the accompanying drawings. In the drawings, the same reference numerals will be used throughout to designate the same or equivalent elements. In addition, a detailed description of well-known features or functions will be ruled out in order not to unnecessarily obscure the gist of the present disclosure.

Advantages and features of the present disclosure and a method of achieving the same will become apparent with reference to the embodiments described below in detail in conjunction with the accompanying drawings.

However, the present disclosure is not limited to embodiments disclosed below but will be implemented in various different forms, and only these embodiments are provided so that the disclosure of the present disclosure will be thorough and complete and will fully convey the scope of the present disclosure to those skilled in the art to which the present disclosure pertains, and the present disclosure is defined by the scope of the claims.

In addition, in describing the present disclosure, the detailed description of a related known technique or the like will be omitted when it obscures the gist of the present disclosure.

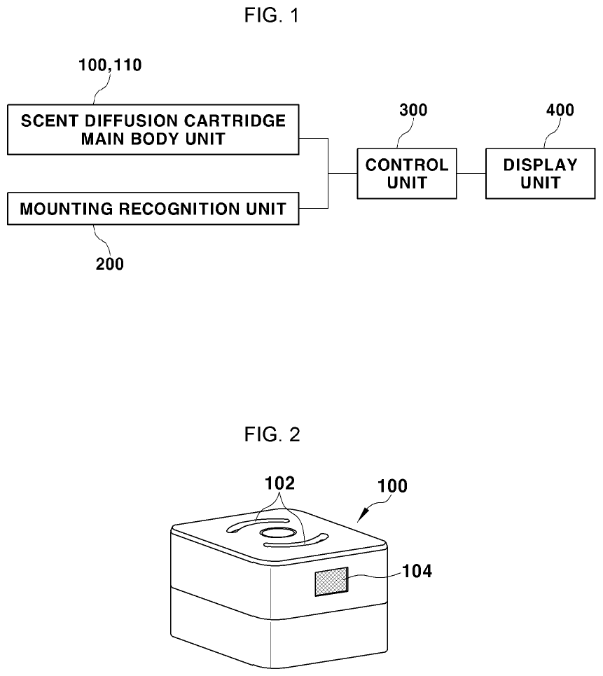

FIG. 1 is a diagram showing a configuration of a scent diffusion cartridge conversion system for a vehicle according to an exemplary embodiment of the present disclosure, and FIG. 2 is a diagram showing a scent diffusion cartridge main body unit of the scent diffusion cartridge conversion system for a vehicle according to an exemplary embodiment of the present disclosure.

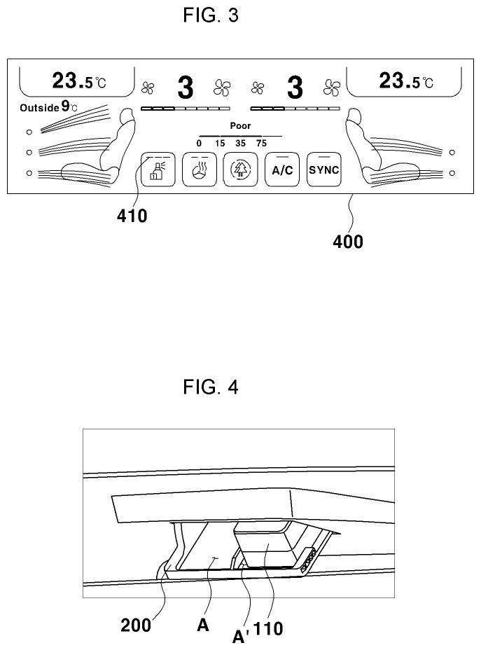

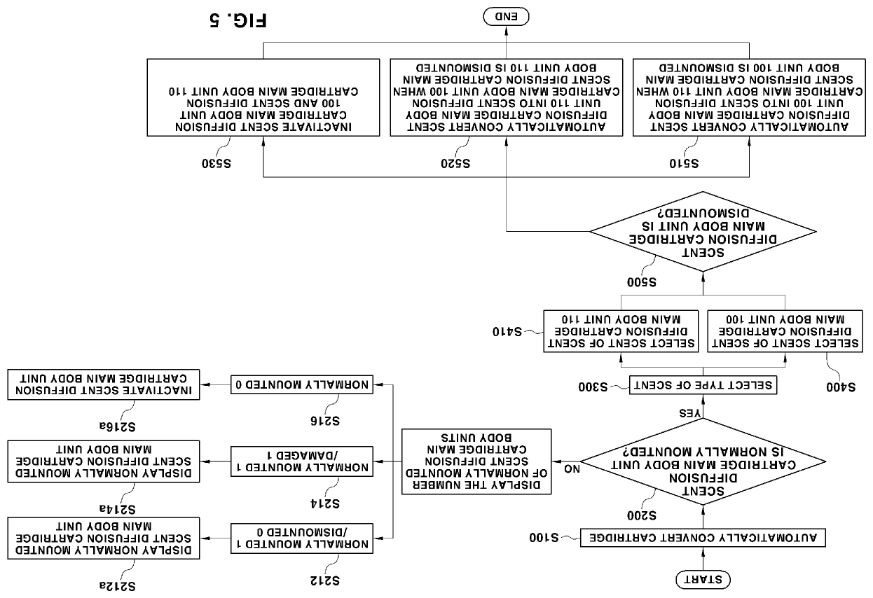

In addition, FIG. 3 is a diagram showing a display unit of the scent diffusion cartridge conversion system for a vehicle according to an exemplary embodiment of the present disclosure, and FIG. 4 is a diagram schematically showing the scent diffusion cartridge conversion system for a vehicle according to an exemplary embodiment of the present disclosure.

As shown in FIG. 1, a scent diffusion cartridge conversion system for a vehicle according to the present embodiment may comprise a scent diffusion cartridge main body unit 100, a mounting recognition unit 200, and a control unit 300.

First, as shown in FIG. 2, the scent diffusion cartridge main body unit 100 may be formed with a plurality of vent holes 102 configured to introduce and discharge air, and as described above, the air may be introduced and discharged through the vent hole 102 to generate scent into the vehicle.

In other words, a plurality of carriers (not shown) configured to retain or emit scent along a flow path of air may be arranged and installed inside the scent diffusion cartridge main body unit 100, and thus the air is introduced through the vent hole 102 to flow and be discharged so that the scent included in the corresponding carrier is diffused into the vehicle.

A plurality of scent diffusion cartridge main body units 100 with different scents are provided, and have the same sizes to be provided to be replaced and mounted, so that different scents are selectively generated into the vehicle when a scent diffusion function is activated by a driver's operation.

As shown in FIG. 4, the mounting recognition unit 200 is provided with a plurality of mounting regions A, A′ in which the scent diffusion cartridge main body unit 100 is mounted, and provided to detect whether the scent diffusion cartridge main body unit 100 is mounted in the mounting regions A, A′.

In addition, the mounting recognition unit 200 may be configured to receive scent information from an IC chip 104 provided in the scent diffusion cartridge main body unit 100 (see FIG. 3), and also recognize different types of scents included in the scent diffusion cartridge main body unit 100 or recognize damage to the IC chip 104 and the like.

Meanwhile, the control unit 300 determines whether the scent diffusion cartridge main body unit 100 is mounted in the plurality of mounting regions A, A′ through the information detected by the mounting recognition unit 200.

Here, as the scent diffusion cartridge main body unit 100 is dismounted from the mounting region A, or damaged, the control unit 300 may be configured to control the scent of the scent diffusion cartridge main body unit 110 mounted in the other mounting region A′ located adjacent to the mounting region A to be selectively diffused.

More specifically, for example, when it is determined that the scent diffusion cartridge main body unit 100 mounted in the mounting region A is dismounted or damaged through the mounting recognition unit 200 provided with the two mounting regions A, A′ (see FIG. 4), the control unit 300 may be configured to control the scent diffusion to be automatically converted into the type of the scent included in the scent diffusion cartridge main body unit 110 mounted in the adjacent mounting region A′.

At this time, as described above, it is preferable that the scent diffusion intensity according to the automatic conversion into the different type of scent is the same as the scent diffusion intensity of the previous scent diffusion cartridge main body unit 100, and in addition, when the scent diffusion intensity is displayed, the scent diffusion intensity may be controlled to be provided to a user through a scent diffusion intensity display portion 410 of the display unit 400 (see FIG. 3).

Meanwhile, when the user arbitrarily dismounts the scent diffusion cartridge main body unit 100 using the features of the above-described configuration, the scent diffusion cartridge main body unit 110 mounted in the adjacent mounting region A′ other than the corresponding scent diffusion cartridge main body unit 100 is activated through the mounting recognition unit 200, so that the control unit 300 may be configured to control the conversion to be performed so that the corresponding scent is diffused, and since the previous scent diffusion cartridge main body unit 100 mounted in the mounting region A is in the inactivated state, that is, the dismounted state on the display unit 400, the selection by the user is impossible, and thus the conversion into the corresponding scent may be difficult before replacement and installation.

Accordingly, when the current scent of the mounted scent diffusion cartridge main body unit 100 mounted as described above is intended to be converted into a different scent, the control unit 300 may be configured to automatically convert the current scent into a different scent of the scent diffusion cartridge main body unit 100 only when the currently operating scent diffusion cartridge is removed even without a separate operation of the audio video navigation (AVN) for changing, thereby providing convenience according to the conversion of the scent diffusion cartridge to the driver while traveling.

More specifically, for example, when a user who does not prefer the scent of the scent diffusion cartridge main body unit 100 gets on the vehicle, or when it is determined that the selected scent is not diffused, the scent may be converted into a scent included in the other cartridge main body unit 110 mounted in the mounting region A′ and diffused only when the scent diffusion cartridge main body unit 100 is dismounted, thereby minimizing the operation according to the scent conversion.

Here, while it has been described above that as the scent diffusion cartridge main body unit 100 mounted in the mounting region A is detached, the conversion into the scent diffusion cartridge main body unit 110 mounted in the other adjacent mounting region A′ is performed, for example, the control unit 300 may be configured to also perform the scent conversion into the scent diffusion cartridge main body unit 110 mounted in the other adjacent mounting region A′ when the IC chip 104 of the scent diffusion cartridge main body unit 100 is damaged and thus the scent diffusion cartridge main body unit 100 may not be recognized (see FIG. 2).

Meanwhile, the control unit 300 may be configured to control the scent information of the scent diffusion cartridge main body units 100, 110 mounted in the mounting regions A, A′ to be displayed on the display unit 400 according to the type of scent, respectively (see FIG. 1), and may be configured to control the scent diffusion cartridge main body units 100, 110 to be selectively converted by the user's operation.

In other words, when each of the scent diffusion cartridge main body units 100, 110 mounted in the two mounting regions A, A′ are detected as corresponding to the normally mounted state, as shown in FIG. 3, the control unit 300 may be configured to allow the scent information of the two scent diffusion cartridge main body units 100, 110 generating different scents to be displayed on the display unit 400 together, thereby selectively converting the scent diffusion cartridge main body unit 100 by the user's operation.

In addition, when the scent diffusion cartridge main body units 100, 110 mounted in the two mounting regions A, A′ are each converted from the normally mounted state into the dismounted state, the control unit 300 may be configured to control the selection of the scent diffusion cartridge main body unit 100 itself to be inactivated on the display unit 400.

This is to control the scent diffusion intensity to be OFF, and at the same time, also control the selection of the scent diffusion cartridge main body unit 100 to be inactivated on the display unit 400 when all of the plurality of scent diffusion cartridge main body units 100, 110 are converted into the dismounted state (or damaged state), thereby preventing the occurrence of the unnecessary selection operation by the user in advance, and effectively transmitting the fact indicating that the mounted state of the scent diffusion cartridge main body unit 100 is not a normal state to the user through the inactivation of the selection.

In addition, in detecting the mounted state of the scent diffusion cartridge main body unit 100, when it is detected that the scent diffusion cartridge main body units 100, 110 are not mounted in both the mounting regions A, A′, more specifically, they are all in the dismounted state or in a state in which any one of the two is dismounted, the control unit 300 may be configured to control the number of mounted scent diffusion cartridge main body units 100 to be selectively displayed on the display unit 400, and may be configured to control the selection of only the scent diffusion cartridge main body unit 100 corresponding to the mounted state to be activated.

For example, when it is determined that the scent diffusion cartridge main body units 100, 110 are not normally mounted in the mounting regions A, A′, the control unit 300 may be configured to first control the number of the scent diffusion cartridge main body units to be displayed on the display unit 400, and may be configured to control the corresponding scent to be diffused by allowing the selection of the scent diffusion cartridge main body unit 100 corresponding to the normally mounted state to be activated when the number is indicated by 1.

In addition, when the number of normally mounted scent diffusion cartridge main body units is indicated by 0 on the display unit 400, this is in the state in which the scent diffusion cartridge main body unit 100 is not normally mounted in both the mounting regions A, A′, that is, both the scent diffusion cartridge main body units 100, 110 are dismounted from the mounting regions A, A′, so that the control unit 300 may be configured to control the selection of the scent diffusion cartridge main body unit 100 on the display unit 400 itself to be inactivated.

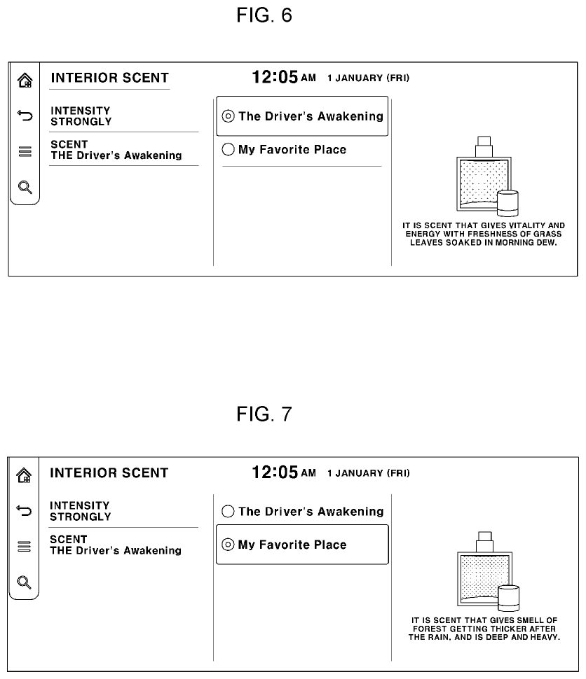

Hereinafter, FIG. 5 is a diagram sequentially showing a scent diffusion cartridge conversion method for a vehicle according to another embodiment of the present disclosure, and FIG. 6 is a diagram showing a selection screen of the scent diffusion cartridge main body unit in the scent diffusion cartridge conversion method for a vehicle according to another embodiment of the present disclosure.

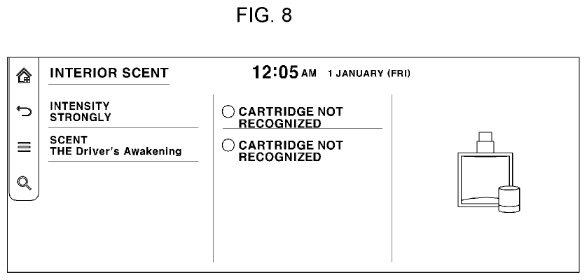

In addition, FIG. 7 is a diagram showing another selection screen of the scent diffusion cartridge main body unit in the scent diffusion cartridge conversion method for a vehicle according to another embodiment of the present disclosure, and FIG. 8 is a diagram showing a screen in which the selection of the scent diffusion cartridge main body unit in the scent diffusion cartridge conversion method for a vehicle according to another embodiment of the present disclosure is inactivated.

As shown in FIG. 5, a scent diffusion cartridge conversion method for a vehicle according to the present embodiment will be sequentially described as follows.

First, in allowing the scents of the plurality of scent diffusion cartridge main body units, for example, the two scent diffusion cartridge main body units 100, 110 including different scents to be automatically converted (S100), it is determined whether the scent diffusion cartridge main body units 100, 110 mounted in the plurality of mounting regions A, A′ are each in the normally mounted state (S200).

At this time, as it is determined that both the scent diffusion cartridge main body units 100, 110 mounted in each of the mounting regions A, A′ are normally mounted, the type of scent of any one scent diffusion cartridge main body unit of the plurality of scent diffusion cartridge main body units 100, 110 is controlled to be selected by the user's operation through the display unit 400 (S300).

Here, in performing the above selection, when the type of scent of the scent diffusion cartridge main body unit 100 is selected by the user (S400), as shown in FIG. 6, the description of the type of corresponding scent may be controlled to be displayed on the display unit 400.

Likewise, when the type of scent of the scent diffusion cartridge main body unit 110 is selected by the user (S410), as shown in FIG. 7, the description of the type of corresponding scent may be controlled to be displayed on the display unit 400.

Thereafter, as it is continuously determined whether the scent diffusion cartridge main body units 100, 110 are dismounted respectively, and as it is determined that the scent diffusion cartridge main body units 100, 110 in the mounted state are dismounted or damaged, the scents of the scent diffusion cartridge main body units 100, 110 mounted in the adjacent mounting regions A, A′ are controlled to be diffused.

In other words, when it is determined that the scent diffusion cartridge main body unit 100 is converted from the state of being mounted in the mounting region A into the dismounted state (or damaged state), the control unit 300 may be configured to control the automatic conversion into the scent diffusion cartridge main body unit 110 mounted in the other mounting region A′ to be performed (S510).

More specifically, for example, when it is determined that the scent diffusion cartridge main body unit 100 mounted in the mounting region A of the scent diffusion cartridge main body units 100, 110 mounted in the two mounting regions A, A′ is dismounted (or damaged), the control unit 300 may be configured to control the scent diffusion function of the scent diffusion cartridge main body unit 110 mounted in the mounting region A′ to be activated, so that the scent with the type of scent included in the scent diffusion cartridge main body unit 110 may be diffused.

Likewise, when it is determined that the scent diffusion cartridge main body unit 110 is converted from the state of being mounted in the mounting region A′ into the dismounted state in the state in which any one scent of the scent diffusion cartridge main body unit 110 is selected (S410), the control unit 300 may be configured to control the automatic conversion into the scent of the scent diffusion cartridge main body unit 100 mounted in the mounting region A to be performed (S520).

In other words, when it is determined that the scent diffusion cartridge main body unit 110 mounted in the mounting region A′ is dismounted (or damaged), the control unit 300 may be configured to control the scent function of the scent diffusion cartridge main body unit 100 mounted in the mounting region A to be activated, so that the scent with the type of scent included in the scent diffusion cartridge main body unit 100 may be diffused.

In addition, when it is determined that both the two scent diffusion cartridge main body units 100, 110 are dismounted from the mounting regions A, A′, respectively, as shown in FIG. 8, the control unit 300 may be configured to control the selection of the scent diffusion cartridge main body units 100, 110 itself to be all inactivated on the display unit 400 (S530).

As a result, when both the two scent diffusion cartridge main body units 100, 110 are converted from the normally mounted state into the dismounted state (or damaged state), the control unit 300 may be configured to control the scent intensity to be OFF, and also control the selection of the scent diffusion cartridge main body unit 100 to be all inactivated on the display unit 400, thereby preventing the occurrence of the unnecessary selection operation in advance, and effectively transmitting the fact that the mounted states of the scent diffusion cartridge main body units 100, 110 are not the normal state to the user through the inactivation of the selection.

Meanwhile, as a result of determining whether the scent diffusion cartridge main body units 100, 110 mounted in the plurality of mounting regions A, A′ are in the normally mounted state respectively (S200), the control unit 300 may be configured to control the number of scent diffusion cartridge main body units to be displayed when it is determined that the scent diffusion cartridge main body units are not all in the normally mounted state (S210).

In the operation of determining the number of mounted scent diffusion cartridge main body units, as the number of scent diffusion cartridge main body units 100, 110 mounted in the mounting regions A, A′ is indicated by, for example, 1 other than 0, thereby figuring out its cause and displaying the same.

In other words, when the number of normally mounted scent diffusion cartridge main body units is indicated by 1, it is first determined whether the scent diffusion cartridge main body units 100, 110 are mounted in the mounting regions A, A′, respectively, through the mounting recognition unit 200, but when it is determined that the scent diffusion cartridge main body units 100 is normally mounted only in one mounting region A of the two mounting regions A, A′ (S212), only the scent information of the normally mounted scent diffusion cartridge main body unit 100 is displayed on the display unit 400 in order to display the scent intensity and the like (S212A).

In addition, when the number of normally mounted scent diffusion cartridge main body units is indicated by 1, it is determined whether the scent diffusion cartridge main body units 100, 110 are normally mounted in the mounting regions A, A′, respectively, but when it is determined that the scent diffusion cartridge main body unit 100 is mounted in one mounting region A, but the scent diffusion cartridge main body unit 110 is damaged in the other mounting region A′ (S214), only the scent information of the normally mounted scent diffusion cartridge main body unit 100 is displayed on the display unit 400 in order to display the scent intensity and the like (S214A).

For example, this is to determine that the scent diffusion cartridge main body unit 110 is damaged when recognition is impossible as the IC chip 104 of the scent diffusion cartridge main body unit 110 is damaged and the like, and allow only the scent information of the normally mounted scent diffusion cartridge main body unit 100 to be displayed on the display unit 400.

Meanwhile, in the operation of determining the number of mounted scent diffusion cartridge main body units (S210), as the number of scent diffusion cartridge main body units 100, 110 mounted in the mounting regions A, A′ is indicated by 0 (S216), the selection of the scents of the scent diffusion cartridge main body units 100, 110 is controlled to be inactivated (S216).

In other words, when the number of normally mounted scent diffusion cartridge main body units is indicated by 0, this is in the state in which the scent diffusion cartridge main body units 100, 110 are not normally mounted in both the mounting regions A, A′, that is, the state in which they are damaged, so that as shown in FIG. 8, the control unit 300 may be configured to control the selection of the scent of the scent diffusion cartridge main body unit 100 itself to be inactivated on the display unit 400.

According to the present disclosure, the plurality of scent diffusion cartridges can be provided, but the currently operating scent diffusion cartridge may be automatically converted into the other scent diffusion cartridge mounted when the currently operating scent diffusion cartridge is removed or damaged, for example, when the scent of the current scent diffusion cartridge is intended to be changed, the automatic conversion into the scent of the other scent diffusion cartridge and the change in the type of scent may be performed only when the currently operating scent diffusion cartridge is removed even without operation of the separate audio video navigation (AVN) for changing, thereby providing convenience according to the conversion of the scent diffusion cartridge to the driver while traveling.

In addition, according to the present disclosure, when it is determined that all of the plurality of scent diffusion cartridges are removed or damaged, the scent diffusion intensity can be controlled to be OFF, and at the same time, the selection of the scent diffusion cartridge can also be controlled to be inactivated on the display unit of the vehicle, thereby preventing the occurrence of the unnecessary selection operation in advance.

While the present disclosure has been described above with reference to the embodiment(s) shown in the drawings, this is merely illustrative, and those skilled in the art will understand that the present disclosure may be variously modified therefrom, and all or some of the above-described embodiment(s) may also be selectively combined and configured. Accordingly, the true technical scope of the present disclosure should be defined by the technical spirit of the appended claims.

What is claimed is:

1. A scent diffusion cartridge conversion system for a vehicle, comprising:
a plurality of scent diffusion cartridge main body units configured to generate different scents;
a mounting recognition unit comprising a plurality of mounting regions in which a scent diffusion cartridge main body unit is mounted, configured to detect whether the scent diffusion cartridge main body unit is mounted in a mounting region; and
a control unit configured to:
determine whether the scent diffusion cartridge main body unit is mounted in the mounting region from the mounting recognition unit; and
control a scent of the scent diffusion cartridge main body unit mounted in another mounting region to be selectively diffused as the scent diffusion cartridge main body unit is dismounted from the mounting region.

2. The scent diffusion cartridge conversion system of claim 1, wherein the control unit is configured to:
control scent information of the scent diffusion cartridge main body unit mounted in the mounting region to be each displayed according to a type of scent; and
control the scent information to be selectively convertible by a user's operation.

3. The scent diffusion cartridge conversion system of claim 1, wherein the control unit is configured to control a selection of a corresponding scent diffusion cartridge main body unit dismounted to be inactivated as the scent diffusion cartridge main body unit is converted from a mounted state into a dismounted state in the mounting region.

4. The scent diffusion cartridge conversion system of claim 1, wherein the control unit is configured to control a selection of all of the scent diffusion cartridge main body units to be inactivated as all of the scent diffusion cartridge main body units are converted from a mounted state into a dismounted state in the mounting region.

5. The scent diffusion cartridge conversion system of claim 1, wherein, for scent diffusion cartridge main body units corresponding to a mounted state to be activated, the control unit is configured to control a number of scent diffusion cartridge main body units mounted in the mounting region to be selectively displayed.

6. A method for scent diffusion cartridge conversion for a vehicle, the method comprising:
determining, for a plurality of scent diffusion cartridge main body units mounted in a plurality of mounting regions provided in a mounting recognition unit, whether each of the plurality of scent diffusion cartridge main body units is mounted;
controlling a type of scent of any one scent diffusion cartridge main body unit, of the plurality of scent diffusion cartridge main body units, as all of the scent diffusion cartridge main body units are mounted in a mounting region;
determining whether each of the plurality of scent diffusion cartridge main body units is dismounted; and
controlling a scent of each scent diffusion cartridge main body unit mounted in another mounting region to be diffused as it is determined that a mounted scent diffusion cartridge main body unit is dismounted.

7. The method of claim 6, wherein controlling the scent further comprises controlling a selection of a scent diffusion cartridge main body unit to be inactivated as it is determined that all of the plurality of scent diffusion cartridge main body units are dismounted from the mounting region.

8. The method of claim 6, wherein the determining whether each of the plurality of scent diffusion cartridge main body units is mounted further comprises:
determining a number of mounted scent diffusion cartridge main body units; and
controlling a number of scent diffusion cartridge main body units mounted in the mounting region to be displayed as it is determined that all of the scent diffusion cartridge main body units are not mounted in the mounting region.

9. The method of claim 8, wherein the operation of determining the number of mounted scent diffusion cartridge main body units controls the selection of the scent diffusion cartridge main body unit in a normal state to be selectively activated as it is determined that the number of scent diffusion cartridge main body units mounted in the mounting region is not 0.

10. The method of claim 8, wherein the operation of determining the number of mounted scent diffusion cartridge main body units controls the selection of the scent diffusion cartridge main body unit in a normal state to be inactivated as it is determined that the number of scent diffusion cartridge main body units mounted in the mounting region is 0.
